(19) United States
(12) Patent Application Publication
(10) Pub. No.: US 2021/0076719 A1
(43) Pub. Date: Mar. 18, 2021

(54) COMPOSITION COMPRISING GLUCOSYLATED TERPENE GLYCOSIDES, TERPENE GLYCOSIDES AND CYCLODEXTRINE

(71) Applicant: Firmenich SA, Meyrin (CH)

(72) Inventor: Olivier HAEFLIGER, Geneva (CH)

(73) Assignee: Firmenich SA, Meyrin (CH)

(21) Appl. No.: 16/954,329

(22) PCT Filed: Feb. 25, 2019
(86) PCT No.: PCT/EP2019/054611

§ 371 Date: Jun. 16, 2020

Related U.S. Application Data
(60) Provisional application No. 62/635,023, filed on Feb. 26, 2018.

(30) Foreign Application Priority Data
Jun. 5, 2018 (EP) 18176110.7

(51) Int. Cl. A23L 27/30; A23L 27/00

(52) U.S. Cl. CPC A23L 27/36; A23L 27/86; A23L 27/84

(57) ABSTRACT

The various aspects presented herein relate to processes for preparing food ingredients, flavors and sweeteners from extracts of plants that contain terpene glycosides such as Stevia rebaudiana Bertoni, Rubus suavissimus or Siraitia grosvenorii. Also presented herein are formulations and uses of compositions made from the processes.

### CROSS-REFERENCE TO RELATED APPLICATIONS

The present application claims priority to U.S. Provisional Patent Application Ser. No. 62/635,023, filed on Feb. 26, 2018, and European Patent Application Serial No. 18176110.7, filed on Jun. 5, 2018, the entire contents of which are hereby incorporated by reference in their entirety.

### FIELD OF THE INVENTION

The various aspects presented herein relate to processes for preparing food ingredients, flavors and sweeteners from extracts of plants that contain terpene glycosides such as Stevia rebaudiana Bertoni, Rubus suavissimus or Siraitia grosvenorii. Further provided herein are formulations and uses of compositions made from the processes.

### BACKGROUND

Natural sources of sweet compounds continue to be sought after, as negative health effects generated by the ingestion of sugar by large populations continue to concern health officials and mankind in general. The trends towards obesity and obesity bear this out. The search for sweet plant extracts that have close to zero calorie contribution to a human's diet continues in order to find sweet and flavorful compounds that might replace sugars in order to reduce the instances of obesity, diabetes and cardiovascular diseases.

Terpene glycosides, such as, for example, steviol glycosides from Stevia (Stevia rebaudiana Bertoni) extracts, rubusoside from blackberry leaves (Rubus suavissimus) extracts, and mogrosides from monk fruit (Siraitia grosvenorii) extracts, have been advanced and reported as low calorie alternatives to sugar. However, these products continue to have negative attributes such as bitterness, lingering aftertaste, or licorice flavor. Some of these problems associated with replacing sugar with sweet extracts have been partially addressed by the transglucosylation of terpene glycosides using enzymes. However, many challenges to obtain a low calorie natural sweetener free of undesirable taste attributes still exist.

### SUMMARY

One aspect presented herein provides a composition,
wherein the composition is a flavor and taste modifying composition,
wherein the composition comprises:
   a) glucosylated terpene glycosides of different degrees of glucosylation;
   b) residual terpene glycoside molecules selected from the group consisting of stevioside, rebaudioside A, rebaudioside C, rebaudioside D, other steviol glycosides, rubusoside, mogrosides, and any combination thereof; in an amount ranging from 1% to 40% on a dry basis; and
   c) at least one cyclodextrin.

In one aspect, the glucosylated terpene glycosides comprise glucosylated steviol glycosides.

In one aspect, the glucosylated terpene glycosides comprise glucosylated rubusoside.

In one aspect, the glucosylated terpene glycosides comprise glucosylated mogrosides.

In one aspect, the glucosylated mogrosides comprise glucosylated mogroside V.

In one aspect, the amount of the at least one cyclodextrin in the composition is from 0.001 to 14% w/w.

In one aspect, the amount of the at least one cyclodextrin in the composition is from 0.01 to 14% w/w.

In one aspect, the at least one cyclodextrin comprises 6 to 8 glucose units.

In one aspect, the at least one cyclodextrin comprises 9 to 12 glucose units.

In one aspect, the different degrees of glucosylation comprises from 1 to 20 glucose residues attached to the terpene glycoside via a glucosidic bond.

In one aspect, the residual terpene glycoside molecules comprise steviol glycoside molecules.

In one aspect, the residual terpene glycoside molecules comprise rebaudioside A molecules.

In one aspect, the residual terpene glycoside molecules comprise mogroside molecules.

In one aspect, the mogroside molecules comprise mogroside V molecules.

In one aspect, the residual terpene glycoside molecules comprise rubusoside molecules.

One aspect presented herein provides a method,
wherein the method increases a taste and flavor intensity of a flavored article,
wherein the method comprises adding to the flavored article a composition comprising the composition according to some aspects presented herein, and
wherein increasing the taste and flavor intensity comprises delaying a sweetness onset in the food or beverage product.

One aspect presented herein provides the use of the composition according to some aspects presented herein to enhance, improve, or modify the sweet taste of a flavored article.

One aspect presented herein provides a method,
wherein the method enhances, improves, or modifies the sweet taste of a flavored article,
wherein the method comprises adding the composition according to some aspects presented herein to the flavored article, in an amount effective to enhance, improve, or modify the sweet taste of the flavored article.

### DETAILED DESCRIPTION

In the following description, reference is made to specific embodiments which may be practiced, which is shown by way of illustration. These embodiments are described in detail to enable those skilled in the art to practice the invention described herein, and it is to be understood that other embodiments may be utilized and that logical changes may be made without departing from the scope of the aspects presented herein. The following description of example embodiments is, therefore, not to be taken in a limited sense, and the scope of the various aspects presented herein is defined by the appended claims.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

It has been unexpectedly discovered that glycosylation of terpene glycosides beyond a certain number of glucose units effectively reduces sweetness. It has also been discovered that with the reduction of sweetness, the glucosylated terpene glycosides (hereinafter “GTG”) can contribute to the modification of flavor and sweetness profiles in a wide range of food and beverage applications.

In particular, it has been unexpectedly discovered that glycosylation of terpene glycosides beyond a certain number of glucose units effectively reduces sweetness. It has also been discovered that with the reduction of sweetness, GTG can contribute to the modification of flavor and sweetness profiles in a wide range of food and beverage applications.

It has also been discovered that the modification of flavor and sweetness profile is affected by the composition of the blend of GTG, terpene glycosides and maltodextrin. Furthermore, it has also been discovered that the modification of flavor and sweetness profile is affected by the composition of the blend of GTG, terpene glycosides and at least one cyclodextrin.

Without intending to be limited to any particular theory, the size and amount of GTG molecules, the type and amount of residual terpene glycosides, and the type and amount of the at least one cyclodextrin contribute different degrees of flavor modification. Therefore, while sweetness decreases with glycosylation, flavor modification increases. The flavor modification may include an increase (enhance) in certain flavor note and/or reduction (suppress) of certain other flavor notes. The blend of GTG, terpene glycosides and the at least one cyclodextrin provides a certain amount of sweetness, but the present invention shows that such blends modify the flavor and sweetness profile in a wide range of food and beverage applications.

In one embodiment, the present invention pertains to a process where the blend of specific types and contents of GTG and terpene glycosides is made from an alpha-glycosyl sugar compound selected from the group consisting of maltooligosaccharide, a starch, a partial starch hydrolysate, a cyclodextrin and sucrose; and terpene glycosides found in Stevia rebaudiana Bertoni plants, terpene glycosides found in extracts of Rubus suavissimus plants, terpene glycosides found in Siraitia grosvenorii plants, or mixtures thereof.

In some aspects, the process where the blend of specific types and contents of GTG and terpene glycosides is made from Tapioca starch and stevia extract is described in GB Patent Application Publication No. 2027423A.

In one embodiment, the present invention pertains to a process where the blend of specific types and contents of GTG and terpene glycosides is made from Tapioca starch and terpene glycosides found in Stevia rebaudiana Bertoni plants, terpene glycosides found in extracts of Rubus suavissimus plants, terpene glycosides found in Siraitia grosvenorii plants, or mixtures thereof, which contains total steviol glycoside content from 50 to 100%, with predominant steviol glycoside molecules of stevioside, rebaudioside (Reb) A, Reb B, Reb C and Reb D.

In some aspects, the process where the blend of specific types and contents of GTG and terpene glycosides is made from Tapioca starch and stevia extract is described in U.S. Pat. No. 9,420,815.

The present invention also pertains to the specific composition of blends, where the types and amounts of different GTG molecules and terpene glycoside molecules contribute different degree of taste modification in the food and beverage applications.

One aspect presented herein provides a composition,
wherein the composition is a flavor and taste modifying composition,
wherein the composition comprises:
   a) glucosylated terpene glycosides of different degrees of glucosylation;
   b) residual terpene glycoside molecules selected from the group consisting of stevioside, rebaudioside A, rebaudioside C, rebaudioside D, other steviol glycosides, rubusoside, mogrosides, and any combination thereof; in an amount ranging from 1% to 40% on a dry basis; and
   c) at least one cyclodextrin.

In some aspects, the glucosylated terpene glycosides comprise glucosylated steviol glycosides.

In some aspects, the glucosylated terpene glycosides comprise glucosylated rubusoside.

In some aspects, the glucosylated terpene glycosides comprise glucosylated mogrosides.

In some aspects, the glucosylated mogrosides comprise glucosylated mogroside V.

In some aspects, the amount of the at least one cyclodextrin in the composition is from 0.001 to 14% w/w.

In some aspects, the amount of the at least one cyclodextrin in the composition is from 0.01 to 14% w/w.

In some aspects, the at least one cyclodextrin comprises 6 to 8 glucose units.

In some aspects, the at least one cyclodextrin comprises 9 to 12 glucose units.

In some aspects, the different degrees of glucosylation comprises from 1 to 20 glucose residues attached to the terpene glycoside via glucosidic bonds.

In some aspects, the residual terpene glycoside molecules comprise steviol glycoside molecules.

In some aspects, the residual terpene glycoside molecules comprise rebaudioside A molecules.

In some aspects, the residual terpene glycoside molecules comprise rubusoside molecules.

In some aspects, the residual terpene glycoside molecules comprise mogroside mogroside molecules.

In some aspects, the mogroside molecules comprise mogroside V molecules.

Advantages of the present invention will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

The compositions can be used as flavor and sweetness profile modifier in various food and beverage products. Non-limiting examples of food and beverage products include carbonated soft drinks, ready to drink beverages, energy drinks, isotonic drinks, low-calorie drinks, zero-calorie drinks, sports drinks, teas, fruit and vegetable juices, juice drinks, dairy drinks, yoghurt drinks, alcohol beverages, powdered beverages, bakery products, cookies, biscuits, baking mixes, cereals, confectioneries, candies, toffees, chewing gum, dairy products, flavored milk, yoghurts, flavored yoghurts, cultured milk, soy sauce and other soy base products, salad dressings, mayonnaise, vinegar, frozen-desserts, meat products, fish-meat products, bottled and canned foods, tabletop sweeteners, fruits and vegetables.

The taste and flavor profile of a food or beverage product including a taste and flavor modifying composition, wherein the taste and flavor modifying composition includes the blend of glucosylated terpene glycosides, terpene glycosides and cyclodextrin, may be more intense than a comparative taste and flavor profile of a comparative food or beverage product which does not include the taste and flavor modifying composition. Moreover, the mouthfeel of a food or beverage product including the taste and flavor modifying composition, wherein the taste and flavor modifying composition includes glucosylated terpene glycosides, selected terpene glycosides and cyclodextrins, may be improved in relation to a mouthfeel of a comparative food or beverage product which does not include the taste and flavor modifying composition.

Additionally the compositions can be used in drug or pharmaceutical preparations and cosmetics, including but not limited to toothpaste, mouthwash, cough syrup, chewable tablets, lozenges, vitamin preparations, and the like.

The compositions can be used “as-is” or in combination with other sweeteners, sugar alcohols, flavors and food ingredients in food, beverage, pharmaceutical and cosmetics applications.

The present invention pertains to a process where the blend of specific types and contents of GSG and SG is made from an alpha-glycosyl sugar compound selected from the group consisting of maltooligosaccharide, a starch, a partial starch hydrolysate, a cyclodextrin and sucrose; and terpene glycosides found in Stevia rebaudiana Bertoni plants, terpene glycosides found in extracts of Rubus suavissimus plants, terpene glycosides found in Siraitia grosvenorii plants, or mixtures thereof.

In some aspects, the process where the blend of specific types and contents of GSG and SG is made from Tapioca starch and stevia extract is described in GB Patent Application Publication No. 2027423A.

In an alternate aspect, the present invention pertains to a process where the blend of specific types and contents of GTG and terpene glycoside is made from Tapioca starch and terpene glycosides found in Stevia rebaudiana Bertoni plants, terpene glycosides found in extracts of Rubus suavissimus plants, terpene glycosides found in Siraitia grosvenorii plants, or mixtures thereof, which contains total steviol glycoside content from 50 to 100%, with predominant steviol glycoside molecules of stevioside, rebaudioside (Reb) A, Reb B, Reb C and Reb D.

In some aspects, the process where the blend of specific types and contents of GTG and terpene glycoside is made from Tapioca starch and stevia extract is described in U.S. Pat. No. 9,420,815.

In this disclosure, a process is developed to make a mixture of glucosylated terpene glycosides with a small amount of other terpene glycosides and maltodextrin. Alternatively, in this disclosure, a process is developed to make a mixture of glucosylated terpene glycosides with a small amount of other terpene glycosides and cyclodextrin.

The process involves precise control of pH, temperature, enzyme source and activity, quality of extract, glucose-donor (amount and source) and time to achieve blends of different ratios of glucosylated terpene glycosides, terpene glycoside molecules, and at least one cyclodextrin.

In one aspect, the amount of the at least one cyclodextrin in the composition is from 0.001 to 14% w/w.

In one aspect, the amount of the at least one cyclodextrin in the composition is from 0.01 to 14% w/w.

In one aspect, the at least one cyclodextrin comprises 6 to 8 glucose units.

In one aspect, the at least one cyclodextrin comprises 9 to 12 glucose units.

In some aspects, the present disclosure pertains to an enzymatic process for producing an ingredient containing glucosylated forms of stevioside, Rebaudioside A, Rebaudioside B, Rebaudioside C, Rebaudioside D, Rebaudioside E, Rebaudioside F, dulcoside A, steviolbioside, rubusoside, terpene glycosides found in Stevia rebaudiana Bertoni plants, terpene glycosides found in Rubus suavissimus plants, terpene glycosides found in Siraitia grosvenorii plants, or mixtures thereof.

In some aspects, the starting material for the enzymatic process may be an extract of Stevia rebaudiana Bertoni plants, or, alternatively, an extract of Rubus suavissimus plants, or, alternatively, an extract of Siraitia grosvenorii plants. In some embodiments, the plant extracts contain one, or more than one terpene glycoside.

For example, by way of illustration, Stevia rebaudiana Bertoni, produces a number of diterpene glycosides, including stevioside, Rebaudioside A, Rebaudioside B, Rebaudioside C, Rebaudioside D, Rebaudioside E, Rebaudioside F, dulcoside A, and steviolbioside. As another example, rubusoside may be obtained from, but not limited to blackberry leaves (Rubus suavissimus), containing substantially a single terpene glycoside called rubusoside. Rubusoside may also be found in low amounts in stevia leaves.

Alternatively, the starting material for the enzymatic process may be a terpene glycoside purified from either an extract of Stevia rebaudiana Bertoni plants, or an extract of Rubus suavissimus plants, or an extract of Siraitia grosvenorii plants.

In some aspects, the terpene glycoside starting material is selected from the group consisting of: stevioside, Rebaudioside A, Rebaudioside B, Rebaudioside C, Rebaudioside D, Rebaudioside E, Rebaudioside F, dulcoside A, steviolbioside, rubusoside, mogroside V, and mixtures thereof.

In some aspects, the terpene glycoside starting material is selected from the group consisting of: stevioside, Rebaudioside A, Rebaudioside B, Rebaudioside C, and rubusoside.

In some aspects, the terpene glycoside starting material is the starting material disclosed in U.S. Pat. No. 8,257,948B1.

In some aspects, the terpene glycoside starting material is the starting material disclosed in International Patent Application Publication No. WO 2017/089444A1.

In some aspects, the terpene glycoside starting material is the starting material disclosed in International Patent Application Publication No. WO 2013/019050A1.

In some aspects, the terpene glycoside starting material is the starting material disclosed in European Patent Application Publication No. EP 3003058A1.

The present invention also pertains to the specific composition of blends, where the types and amounts of different GTG molecules and TG molecules contribute different degree of taste modification in the food and beverage applications.

One aspect presented herein provides a method,
wherein the method increases the taste and/or flavor intensity of a flavored article,
wherein the method comprises adding to the flavored article a composition comprising the composition according to some aspects presented herein, and
wherein increasing the taste and flavor intensity comprises delaying a sweetness onset in the food or beverage product.

One aspect presented herein provides the use of the composition according to some aspects presented herein to convey, enhance, improve, or modify a sweet taste of a flavored article.

One aspect presented herein provides a method,
wherein the method conveys, enhances, improves, or modifies a sweet taste of a flavored article,
wherein the method comprises adding the composition according to some aspects presented herein to the flavored article, in an amount effective to convey, enhance, improve, or modify the sweet taste of the flavored article.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

1. A composition,
wherein the composition is a flavor and taste modifying composition,
wherein the composition comprises:
a) glucosylated terpene glycosides of different degrees of glucosylation;
b) residual terpene glycoside molecules selected from the group consisting of stevioside, rebaudioside A, rebaudioside C, rebaudioside D, other steviol glycosides, rubusoside, mogrosides, and any combination thereof; in an amount ranging from 1% to 40% on a dry basis; and
c) at least one cyclodextrin.

2. The composition of claim 1, wherein the glucosylated terpene glycosides comprise glucosylated steviol glycosides.

3. The composition of claim 1, wherein the glucosylated terpene glycosides comprise glucosylated rubusoside.

4. The composition of claim 1, wherein the glucosylated terpene glycosides comprise glucosylated mogrosides.

5. The composition of claim 1, wherein the glucosylated mogrosides comprise glucosylated mogroside V.

6. The composition of claim 1, wherein the amount of the at least one cyclodextrin in the composition is from 0.001 to 14% w/w.

7. The composition of claim 1, wherein the amount of the at least one cyclodextrin in the composition is from 0.01 to 14% w/w.

8. The composition of claim 1, wherein the different degrees of glucosylation comprises from 1 to 20 glucose residues attached to the terpene glycoside via a glucosidic bond.

9. The composition of claim 1, wherein the residual terpene glycoside molecules comprise steviol glycoside molecules.

10. The composition of claim 1, wherein the residual terpene glycoside molecules comprise rebaudioside A molecules.

11. The composition of claim 1, wherein the residual terpene glycoside molecules comprise rubusoside molecules.

12. The composition of claim 1, wherein the residual terpene glycoside molecules comprise mogroside molecules.

13. The composition of claim 1, wherein the mogroside molecules comprise mogroside V molecules.

14. The composition of claim 1, wherein the at least one cyclodextrin comprises 6 to 8 glucose units.

15. The composition of claim 1, wherein the at least one cyclodextrin comprises 9 to 12 glucose units.

16. A use of the composition of claim 1 to convey, enhance, improve, or modify a sweet taste of a flavored article.

17. A method,
wherein the method increases the taste and/or flavor intensity of a flavored article,
wherein the method comprises adding to the flavored article the composition of claim 1, and
wherein increasing the taste and flavor intensity comprises delaying a sweetness onset in the food or beverage product.

18. A method,
wherein the method conveys enhances, improves, or modifies a sweet taste of a flavored article,
wherein the method comprises the composition of claim 1 to the flavored article, in an amount effective to convey enhance, improve, or modify the sweet taste of the flavored article.
